(19) United States
(12) Patent Application Publication
(10) Pub. No.: US 2009/0087884 A1
(43) Pub. Date: Apr. 2, 2009

(54) Microfluidic nucleic acid amplification and separation

(76) Inventors: Timothy Beerling, San Francisco, CA (US); Magdalena Bynum, San Jose, CA (US); Brian Peter, Los Altos, CA (US); Marc Valer Serra, San Francisco, CA (US); Hui Wang, Cupertino, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/906,076

(22) Filed: Sep. 27, 2007

(51) Int. Cl. C12P 19/34; C12M 1/00

(52) U.S. Cl. 435/91.2; 435/287.2

(57) ABSTRACT

Microfluidic devices designed for assaying biochemical molecules are disclosed. The microfluidic devices are capable of assaying nucleic acids for identification of nucleic acid species. The microfluidic devices are adapted to carry out an amplification of the nucleic acid and subsequent separation of amplified nucleic acid species. Also disclosed is a method for amplifying and separating a nucleic acid sample on a microfluidic device.

### BACKGROUND

Microfluidic device technologies, also referred to as lab-on-a-chip technologies, have been proposed for a number of different applications in various fields. In the field of biology, for example, microfluidic devices may be used to carry out cellular assays. In addition, microfluidic devices have been proposed to carry out separation techniques in the field of analytical chemistry. Microfluidic technology is used in systems that perform chemical and biological analysis, as well as chemical synthesis, on a much smaller scale than previous laboratory equipment and techniques. Microfluidic systems offer the advantages of only requiring a small sample of analyte or reagent for analysis or synthesis, and dispensing a smaller amount of waste materials. A typical microfluidic channel or chamber of a microfluidic system has at least one cross-sectional dimension in the range of approximately 0.1 micrometers to 1000 micrometers. Since microfluidic technologies involve the use of small volumes of fluids, microfluidic technologies are particularly desirable in applications that involve fluids that are extremely rare and/or expensive.

One useful chromatographic function served by microfluidic devices is the separation of nucleic acids, e.g. DNA or RNA. Nucleic acids that have been denatured, e.g. by heating, can be separated using electrophoretic chromatography. Commonly used chromatography methods include applying a nucleic acid sample to an agarose gel column and applying a voltage across the column, producing movement of the fragments according to size and charge.

In order for separation of nucleic acids to provide a sufficient amount of one or more target species for detection by an analyzer, nucleic acids are typically amplified prior to analysis. This is true even for separations performed by a microfluidic device. Amplification may be required because, for example, a subsequent technique requires a pool of nucleic acid molecules isolated from a particular nucleic acid fragment. Amplification enables widespread use of such techniques by providing a larger sample for separation and identification of nucleic acid species.

Polymerase chain reaction (PCR) is a powerful technique allowing amplification and analysis of minute quantities of nucleic acids. PCR is a biochemistry and molecular biology technique for isolating and exponentially amplifying a fragment or sequence of interest of a nucleic acid, via enzymatic replication, without using a living organism (such as E. coli or yeast). As PCR is an in vitro technique, it can be performed without restrictions on the form of nucleic acid, and it can be extensively modified to perform a wide array of genetic manipulations. In a conventional laboratory scale reaction, PCR is carried out in reaction tubes that are inserted into a thermal cycler. This is a machine that heats and cools the reaction tubes within it to the precise temperature required for each step of the reaction.

In practice, PCR can fail for various reasons, in part due to its sensitivity to contamination causing amplification of spurious nucleic acid products. Because of this, a number of techniques and procedures have been developed for optimizing PCR conditions. Contamination with extraneous nucleic acid is addressed with lab protocols and procedures that separate pre-PCR reactions from potential nucleic acid contaminants. This usually involves spatial separation of PCR-setup areas from areas for analysis or purification of PCR products, and thoroughly cleaning the work surface between reaction setups. Conventional laboratory scale PCR, when used in preparation for microfluidic separation and analysis, requires additional time to prepare the sample for application to the microfluidic device. More sample, including both nucleic acid and the expensive reagents and materials required to run the PCR reaction, are used in a lab scale PCR than is required for microfluidic analysis. Lab scale PCR requires special thermal cyclers. These thermal cyclers require appreciable time to preheat to the temperatures required to carry out the PCR reaction and, due to their size, can take appreciable time to reach the target temperature in each step of the PCR cycle. Additionally, the need to perform the PCR reaction on a lab scale means that only a finished, amplified product is applied to the separation device. Real time PCR amplification data cannot be measured when PCR is done separately and prior to loading a sample onto a microfluidic device.

### BRIEF DESCRIPTION OF THE DRAWINGS

FIG. 1 is a plan view showing an embodiment of a microfluidic nucleic acid assay device.

FIG. 2 is a plan view showing a second embodiment of a microfluidic nucleic acid assay device.

FIG. 3 is a flowchart showing one embodiment of a method of assaying nucleic acids.

FIGS. 4A-4C are cross-sectional views showing respective locations of the thermal control device in one embodiment of a microfluidic nucleic acid assay device.

FIGS. 5A and 5B are cross-sectional views showing respective locations of the thermal control device in another embodiment of a microfluidic nucleic acid assay device.

FIG. 6 is a cross-sectional view showing an embodiment of a microfluidic nucleic acid assay device incorporated into a system.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

A microfluidic nucleic acid assay device includes features suitable for providing both amplification and separation of nucleic acid species on a single device. Advantages of the device, compared to conventional PCR followed by microfluidic separation, include smaller required sample size, greater accuracy and greater reproducibility of data due to decreased contamination and decreased statistical error introduced by additional handling of samples, and decreased time to process the sample.

Combining nucleic acid amplification in combination with microfluidic separation on a microfluidic device presents several surprising and unexpected advantages to the nucleic acid assay when compared to traditional PCR amplification followed by nucleic acid separation. The ability to use very small volumes of reagents is significant, especially when the reagents are rare or expensive. The heating of a small sample is faster and uses less energy than heating conventional lab scale samples. The reduced time spent changing the temperature during the amplification additionally provides increased accuracy and precision of the temperature profile overall, which in turn reduces unwanted enzyme activities during amplification, for example during ramping up of temperature. An additional benefit of the reduced time spent changing temperature during amplification is that the nucleic acid sample is exposed to heat for less time overall. This results in less degradation of both sample and reagent enzymes, which in turn leads to less waste and an increase in accuracy and precision in quantification of the sample. The handling and transfer of the sample between vessels is obviated, further reducing statistical error in the overall experiment and eliminating a source of sample contamination.

Providing for PCR and separation on a single microfluidic device enables a sample to be separated in steps during the amplification process. For example, between each heating cycle of the PCR amplification, a valve can be briefly opened to allow for a small volume of sample to leave the reaction region and be urged toward the nucleic acid separation region. In this way, real-time monitoring of the PCR reaction can be performed. A significant advantage of this embodiment is the ability to monitor the length of the amplified nucleic acid sample in real time, which is not possible employing other techniques. These additional data enables a user to differentiate specific amplification products of the correct length from nonspecific amplification products of a different length.

Additional advantages and novel features of the invention will be set forth in part in the description that follows, and in part will become apparent upon examination of the following, or may be learned through routine experimentation upon practice of the invention.

In an embodiment, a microfluidic nucleic acid assay device includes a substrate defining features. The features include a sample well, a reaction region in fluid connection to the sample well, a thermal control device positioned proximal to the reaction region, a nucleic acid separation region in fluid communication with the reaction region, a first valve disposed between the well and the reaction region, and a second valve disposed between the reaction region and the nucleic acid separation region. The features collectively occupy an area of the substrate of about 0.1 to 10 cm².

The thermal control device is situated proximal to the reaction region and heats a nucleic sample in the reaction region. In some embodiments, the thermal control device is embedded within the microfluidic nucleic acid assay device. In other embodiments the thermal control device is external to the microfluidic nucleic acid assay device but situated nearby so that it can heat a nucleic acid sample in the reaction region.

In some embodiments, the thermal control device is embedded within the microfluidic nucleic acid assay device itself. In some such embodiments, the thermal control device is situated so as to be in direct contact with a nucleic acid sample within the reaction region. In other embodiments, the thermal control device is situated within the microfluidic nucleic acid assay device but not such that has direct contact with the nucleic acid sample within the reaction region. In some embodiments where the thermal control device is proximate to, but not in direct contact with, the nucleic acid sample within the reaction region, the thermal control device is situated about 25 μm to 500 μm from a bottom portion of the reaction region. In other embodiments, the thermal control device is situated about 100 μm from a bottom portion of the reaction region. In some embodiments, the thermal control device is situated in the side of the substrate opposite the side in which the features are defined. Such a configuration thus defines the spacing between the thermal control device and the reaction region, and also provides convenient access to the thermal control device such that a source of electricity can be easily connected to it.

In some embodiments the thermal control device embedded in the microfluidic nucleic acid assay device is in the form of a ring that surrounds the reaction region. In other embodiments the embedded thermal control device is in the form of a cylinder having one closed end, such that the thermal control device encloses the reaction region. In other embodiments the embedded thermal control device is circular, oval, or rectilinear in shape and is situated immediately below the reaction region. In some embodiments, an embedded thermal control device matches one or more dimensions of the reaction region. In other embodiments the embedded thermal control device is smaller than one or more dimensions of the reaction region. In still other embodiments the embedded thermal control device is larger than one or more dimensions of the reaction region.

In some embodiments, the thermal control device is situated external to the microfluidic nucleic acid assay device. In some embodiments, such external thermal control device is located adjacent the side of the microfluidic nucleic acid assay device substrate in which the reaction region is defined. In other embodiments, the external thermal control device is located adjacent the side of the microfluidic nucleic acid assay device substrate in which the reaction region is defined. In still other embodiments, the external thermal control device is located beside the reaction region.

In some embodiments, a recess is defined in the microfluidic nucleic acid assay device substrate in the side of the microfluidic nucleic acid assay device substrate opposite the side in which the reaction region is defined and proximate to the reaction region. The recess is shaped to receive a thermal control device. In these embodiments, when the microfluidic nucleic acid assay device is situated within a machine having a thermal control device, the thermal control device is external to the microfluidic nucleic acid assay device. The external thermal control device is situated within the recess proximate to the reaction region. In some embodiments, the external thermal control device is a thermoelectric device. In other embodiments, the external thermal control device is a resistive device (joule heater). In some embodiments, the thermal control device is a microheater chip. In other embodiments, the external thermal control device is an electromagnetic heating device, for example, an infrared emitting device or a microwave emitting device, that is disposed above or below the side of the microfluidic nucleic acid assay device substrate in which the sample well is defined. Where the electromagnetic heating device is a microwave emitting device, water present in the nucleic acid sample converts microwave radiation emitted by the microwave emitting device to heat. Where the electromagnetic heating device is an infrared emitting device, the microfluidic nucleic acid assay device additionally includes a structure, such as a black body, that absorbs the infrared radiation emitted by the infrared emitting device and converts the absorbed radiation into heat. One example of a suitable infrared emitting device is an infrared laser. Additionally or alternatively, the electromagnetic heating device may emit electromagnetic energy in other regions of the electromagnetic spectrum, e.g., visible light.

In some embodiments, a nucleic acid sample is loaded onto the sample well of the microfluidic nucleic acid assay device and the sample is urged toward the reaction region. The reaction region is where thermal cycling, such as in a PCR reaction, takes place. The thermal control device is then used to subject a sample within the reaction region to a suitable PCR heating and cooling cycle. The amplified sample is then directed to the nucleic acid separation region. Typically, the sample well is connected by a channel to the reaction region. Typically, the reaction region is connected by a channel to the nucleic acid separation region. Typically, upon at least partly filling the reaction region, a valve situated between the sample well and the reaction region, and another valve situated between the reaction region and the nucleic acid separation region, are closed in order to prevent evaporation or movement of the sample during the amplification. Typically, the nucleic acid separation region is a microfluidic separation column. The nucleic acid separation region can be loaded with a standard electrophoretic separation medium. A voltage applied across the separation column causes the sample to move from the reaction region to the separation column and through the column, separating the individual nucleic acid species. The separated nucleic acid species are typically analyzed as they proceed through the nucleic acid separation region using an external detector to detect the individual nucleic acid species.

The valves employed on the microfluidic nucleic acid assay device may be of a type previously disclosed. For example, mechanical valves may be useful. In embodiments, valves of the microfluidic nucleic acid assay device include a thermoelectric device and a material capable of phase change. Such material, when cooled, becomes solid and blocks the movement of fluid within the microfluidic nucleic acid assay device between the sample well and the reaction region, between the reaction region and the nucleic acid separation region, or both.

The microfluidic nucleic acid assay device can include additional features. For example, additional wells holding reagents for the amplification and separation can be disposed on the microfluidic nucleic acid assay device and connected via channels to the sample well, the reaction region, the nucleic acid separation region, or a combination of these three areas. One or more of these channels can include a valve that controls the timing of addition of reagents to a nucleic acid sample. Multiple sample wells can be connected to multiple reaction regions, which in turn can be connected to multiple nucleic acid separation regions; in this way, multiple separations can be carried out at once without cross contamination. Such a device is useful where a standard nucleic acid is to be separated alongside a sample nucleic acid, or where several nucleic acid samples are advantageously amplified and separated at one time.

The microfluidic nucleic acid assay device can further include one or more layers of a thermoresponsive polymer material disposed over at least a portion of the nucleic acid assay device. The thermoresponsive polymer can be, for example, in a valve region where it functions as the phase changing material. Where present in, for example, the reaction region, the thermoresponsive polymer may melt during heating and mix with a nucleic acid sample present in the reaction region. When cooled, the thermoresponsive polymer could solidify, allowing the remainder of the fluid material containing the nucleic acid to progress toward the nucleic acid separation region.

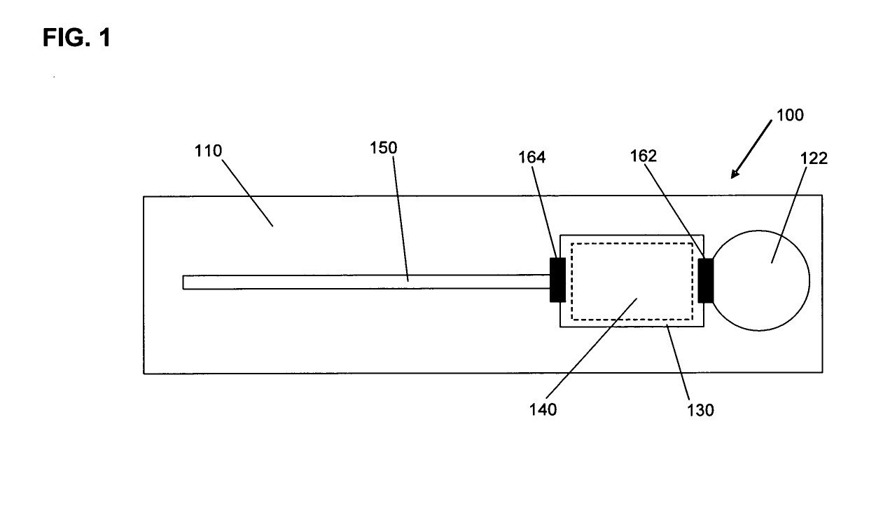

In an embodiment shown in FIG. 1, microfluidic nucleic acid assay device 100 includes a substrate 110, a sample well 122 for receiving a sample, a first valve 162, a reaction region 130 having a thermal control element 140 disposed proximal to reaction region 130, a second valve 164, and a nucleic acid separation region 150. Well 122, valves 162, 164, reaction region 130, and nucleic acid separation region 150 collectively occupy an area of substrate 110 that is about 0.1 to 10 cm². In the example shown, thermal control element 140 is located underneath reaction region 130.

Sample well 122 is in fluid communication with reaction region 130 when first valve 162 is open; similarly, reaction region 130 is in fluid communication with nucleic acid separation region 150 when second valve 164 is open. When valves 162, 164 are closed, reaction region 130 is isolated from both well 122 and nucleic acid separation region 150. Thermal control device 140 changes the temperature of a sample disposed in the reaction region 130 after a sample injected into sample well 122 has traveled to reaction region 130. Where both first valve 162 and second valve 164 are open during the injection, the valves 162, 164 are, in embodiments, closed while thermal control device 140 heats the sample while the sample resides in the reaction region 130.

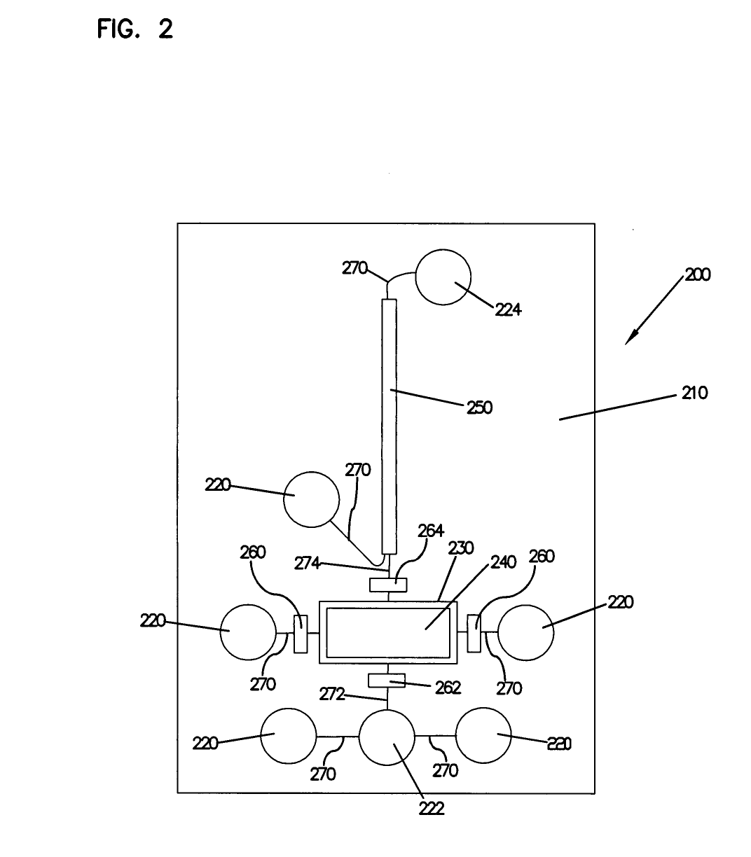

In another embodiment shown in FIG. 2, microfluidic nucleic acid assay device 200 includes a substrate 210, sample well 222 for receiving a sample, first channel 272, first valve 262 disposed in communication with first channel 272, a reaction region 230 having a thermal control element 240 disposed proximal to reaction region 230, a second channel 274, a second valve 264 disposed proximal to second channel 274, and a nucleic acid separation region 250. Sample well 222, channels 272, 274, valves 262, 264, reaction region 230, and nucleic acid separation region 250 collectively occupy an area of substrate 210 of about 0.1 cm² to 10 cm². In this embodiment, thermal control element 240 is located underneath reaction region 230.

Sample well 222 is in fluid communication with reaction region 230 via first channel 272 when first valve 262 is open; similarly, reaction region 230 is in fluid communication with nucleic acid separation region 250 via second channel 274 when second valve 264 is open. When valves 262, 264 are closed, reaction region 230 is isolated from both sample well 222 and separation column 250. Thermal control device 240 heats a sample disposed in the reaction region 230 after one or more samples injected into sample well 222 has traveled to reaction region 230. Where both first valve 262 and second valve 264 are open during the injection, the valves 262, 264 are, in embodiments, closed while thermal control device 240 heats the sample while the sample resides in the reaction region 230.

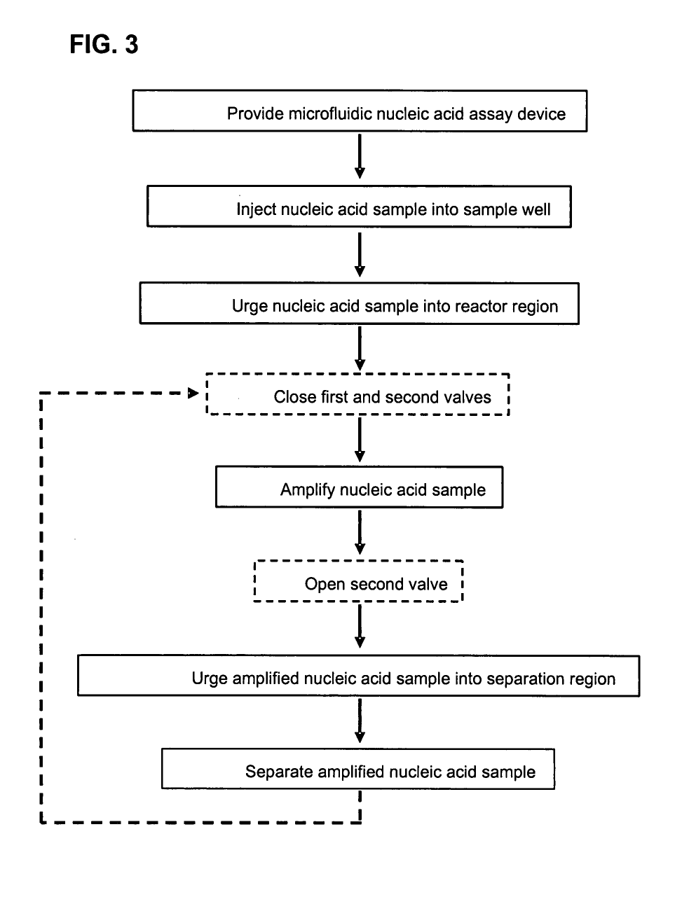

Referring to FIG. 3, a microfluidic nucleic acid assay device according to an embodiment is provided. A nucleic acid sample is injected into the sample well. The nucleic acid sample is urged into reaction region. In some embodiments, the nucleic acid sample is isolated in the reaction region by closing a first and second valve, preventing fluid communication with other features present on the microfluidic nucleic acid assay device. The nucleic acid sample is amplified in the reaction region. In embodiments where the nucleic acid assay device is isolated in the reaction region, the second valve is opened after amplification to provide a fluid connection to the nucleic acid separation region. The amplified nucleic acid sample is urged into the nucleic acid separation region. The amplified nucleic acid sample is separated. In some embodiments, portions of the sample are urged into the reaction region more than one time during the nucleic acid assay. In these embodiments, the amplification, urging, and separation are repeated more than once.

Also shown in microfluidic nucleic acid assay device 200 of FIG. 2 are various additional wells 220, channels 270, and valves 260. Additional wells 200 may have reagents for the PCR and/or separation disposed therein; valves 260 can control the movement of reagents from additional wells 220 through channels 270 and into reaction region 230, nucleic acid separation region 250, or both regions 230, 250. Also shown in microfluidic nucleic acid assay device 200 is a waste well 224, which is disposed to collect separated nucleic acid moving out of nucleic acid separation region 250 after separation of the nucleic acid sample.

In embodiments, in a method of amplifying and separating nucleic acids, a microfluidic nucleic acid assay device 100 is provided. The microfluidic nucleic acid assay device includes a substrate 110 defining features. The features include well 122, reaction region 130 in fluid connection to well 122, thermal control device 140 positioned proximal to the reaction region 130, nucleic acid separation region 150 in fluid communication with the reaction region 130, first valve 162 disposed between the well 122 and the reaction region 130, and second valve 164 disposed between the reaction region 130 and the nucleic acid separation region 150. The features collectively occupy an area of the substrate 110 of about 0.1 to 10 cm². A nucleic acid sample is injected into well 122. The sample is urged into reaction region 130. The nucleic acid is amplified by a polymerase chain reaction within reaction region 130 as facilitated by thermal control device 140. The amplified sample is urged into nucleic acid separation region 150. The amplified nucleic acid sample is separated by eluting the sample through nucleic acid separation region 150.

In embodiments, the method includes injecting about 0.5 μl to 10 μl of a nucleic acid sample into well 122, the nucleic acid sample having about 0.1 g/L to 100 g/L of a nucleic acid in a buffer. In other embodiments, much lower concentrations of nucleic acid sample are used, for example, a single molecule per sample well.

In embodiments, the nucleic acid sample is amplified by a process in which the nucleic acid sample is heated in a first heating of the sample in reaction region 130, 230 to about 94° C. to 96° C. for about 1 to 9 minutes. The nucleic acid sample is annealed by cooling the sample to about 40° C. to 64° C. The cooling is accomplished, in some embodiments, by passive means, e.g. by removing the source of heat from the proximity of the thermal control device, or by removing the source of power from the thermal control device such that is no longer adds heat to the sample. The cooling is accomplished, in other embodiments, by active means, e.g. in embodiments where the thermal control device is a thermoelectric device, reversing the current supply to the thermoelectric device results in active removal of heat from the sample.

The nucleic acid sample is then held at a temperature of about 50° C. to 64° C. for about 20 to 40 seconds. The nucleic acid sample is heated in a second heating to about 65° C. to 75° C. for about 20 to 40 seconds. The nucleic acid sample is then heated in a third heating to about 94° C. to 98° C. for about 20 to 30 seconds. The annealing operation, the holding operation, the second heating operation, and the third heating operation are repeated between 10 and 35 times. Finally, the temperature of the nucleic acid sample is maintained at about 70° C. to 75° C. for about 5 to 15 minutes.

In embodiments, the method includes preparing a nucleic acid sample prior to injecting it onto the microfluidic nucleic acid assay device 100. Such preparation includes, in embodiments, column purification of DNA, desalting of DNA solutions, removal of proteins, removal of RNA, DNA denaturation, buffer exchange, concentration of DNA, or a combination of these. In some embodiments no preparation other than lysing a cell containing a nucleic acid sample is needed.

Substrate

In embodiments, the microfluidic nucleic acid assay device 100 includes substrate 110 which is a substrate known in the art to be suitable for the fabrication of microfluidic devices. Examples of suitable substrate materials include glass, silicon, and plastic. In some embodiments, polydimethylsiloxane (PDMS) is the substrate material. Some advantages of PDMS are that it is very inexpensive, optically clear, and permeable to several substances, including gases. Since air can quickly diffuse out, the latter aspect is very convenient, making it possible to inject fluid into a channel that has no outlet. In other embodiments, poly(ether ether ketone) (PEEK) is the substrate material. Advantages associated with PEEK include excellent mechanical properties and resistance to thermal degradation. Glass and polyimide are other commonly used substrate materials in microfluidic applications.

In some embodiments, the substrate 110 upon which microfluidic features are disposed includes a thermoresponsive polymer. A thermoresponsive polymer is a polymer or blend of polymers having a pronounced temperature-dependent viscosity transition. In embodiments, the thermoresponsive polymers useful in embodiments of the microfluidic nucleic acid assay device include those having a pronounced temperature-dependent viscosity transition within the range of temperatures employed in the amplification. For example, in embodiments, the thermoresponsive polymer is a liquid at all of the temperatures used in the reaction region, for example 35° C. to 105° C., and is a gel at temperatures of less than about 30° C. In these embodiments, the thermoresponsive polymer can be disposed in some or all features defined by substrate 110 of the microfluidic nucleic acid assay device 100. In some such embodiments, the thermoresponsive polymer is disposed over the surface of substrate 110 and within every feature of microfluidic nucleic acid assay device 100. In these embodiments the thermoresponsive polymer is typically a gel in the areas of substrate 110 that are not heated. These embodiments simplify construction of microfluidic nucleic acid assay device 100 and simplify the loading of materials, such as reagents, into features of the substrate 110.

In other embodiments, the pronounced temperature-dependent viscosity transition can occur at temperatures of less than about 22° C. Such embodiments are useful, for example, where a valve employs a thermoelectric element. In such embodiments, the polymer is a liquid, or is otherwise able to flow within features of substrate 110 unless cooled by the thermoelectric element. Upon cooling the viscosity transition, for example from liquid to gel, takes place. As a gel, the thermoresponsive polymer acts as the valve by physically blocking a means of fluid communication between features.

In embodiments, thermoresponsive polymers allow a wider range of viscosities to be accessed in the nucleic acid separation region 150 of microfluidic nucleic acid assay device 100 than is possible using traditional separation techniques. In embodiments, this enables separation of nucleic acids having a broader range of molecular weights and/or molecular dimensions than can be separated using other techniques.

In embodiments, the thermoresponsive polymer or polymer system is poly(ethylene oxide-block-propylene oxide), poly(isopropylacrylamide-graft-poly(ethylene oxide)), poly(acrylamide-graft-poly(isopropylacrylamide)), a blend of hydroxypropylcellulose and hydroxyethylcellulose, a blend of N,N-dialkylacrylamide copolymers, poly(N,N-dimethylacrylamide-graft-poly(ethylene oxide)), or a blend thereof. In embodiments, the thermoresponsive polymer encompasses the entire area of the substrate 110 defining microfluidic features. In other embodiments, the thermoresponsive polymer encompasses only some regions of the substrate 110 defining microfluidic features.

In embodiments, the thermoresponsive polymer has a pronounced temperature-dependent viscosity transition at about 40° to 80° C.; in other embodiments, the thermoresponsive polymer has a pronounced temperature-dependent viscosity transition at about 50° to 70° C.

In an embodiment, in the nucleic acid separation region 150, the thermoresponsive polymer forms a gel which is a separation matrix. In the reaction region 130, the thermoresponsive polymer melts, but will not interfere with the PCR reaction. In another embodiment, the valves 162, 164 or 260 comprise thermoelectric elements disposed proximal to a thermoresponsive polymer, wherein the thermoresponsive polymer is solidified by cooling of the thermoelectric element. In another embodiment, the thermoresponsive polymer is disposed only in the reaction region 130 before introduction of a nucleic acid sample, and the nucleic acid sample is injected into well 122 and urged into the reaction region 130 which contains the thermoresponsive polymer. In this case the nucleic sample requires mechanical or pressure pumping to urge it from well 122 into reaction region 130.

Reaction Region

In embodiments, the microfluidic nucleic acid assay device 100 includes reaction region 130 which is capable of holding a sample having a volume of about 1 picoliter to 25 microliters. In embodiments, the reaction region 130 can be defined by substrate 110 using any technique generally known in the prior art, such as lithographic techniques, laser etching, printing, and microreplication of inverse images of features disposed on PDMS, nickel, or polyimide onto a final substrate such as any of the known thermoplastics. In some embodiments, microreplication is carried out by melt techniques, such as applying heat and pressure to a thermoplastic disposed against a mold having an inverse pattern. In other embodiments, microreplication can be carried out by casting an uncured polymer such as PDMS onto a mold and curing the polymer, followed by removal of the mold.

Additionally, in embodiments, reaction region 130 is etched into a glass substrate employing lithographic techniques. In other embodiments, glass is patterned by photolithography before channels are wet or dry etched. Any of these techniques, or others known in the fabrication of microfluidic devices, can be used to define reaction region 130 on substrate 110 of microfluidic nucleic acid assay device 100.

Thermal Control Device

The thermal control device 140 is situated proximal to reaction region 130 and heats a nucleic acid sample within the reaction region 130 during amplification of the nucleic acid prior to separation. In some embodiments, thermal control device 140 is embedded within the microfluidic nucleic acid assay device 100. In other embodiments thermal control device 140 is external to microfluidic nucleic acid assay device 100 but situated nearby so that it can heat a nucleic acid sample in reaction region 130.

In some embodiments, thermal control device 140 is embedded within the microfluidic nucleic acid assay device 100. In some such embodiments, thermal control device 140 is situated so as to be in direct contact with a nucleic acid sample within reaction region 130. In other embodiments, thermal control device 140 is situated within microfluidic nucleic acid assay device 100 but not in direct contact with the nucleic sample within the reaction region 130. In some embodiments where the thermal control device is proximate to, but not in direct contact with, the nucleic acid sample within reaction region 130, thermal control device 140 is situated about 25 μm to 500 μm from a bottom portion of the reaction region 130. In other embodiments, thermal control device 140 is situated about 100 μm from a bottom portion of reaction region 130. In some embodiments, thermal control device 140 is situated in the side of the substrate 110 opposite the side in which the microfluidic features are defined. Such a configuration defines the spacing between thermal control device 140 and reaction region 130, and also provides convenient access to the thermal control device 140 such that a source of electricity can be easily connected to it.

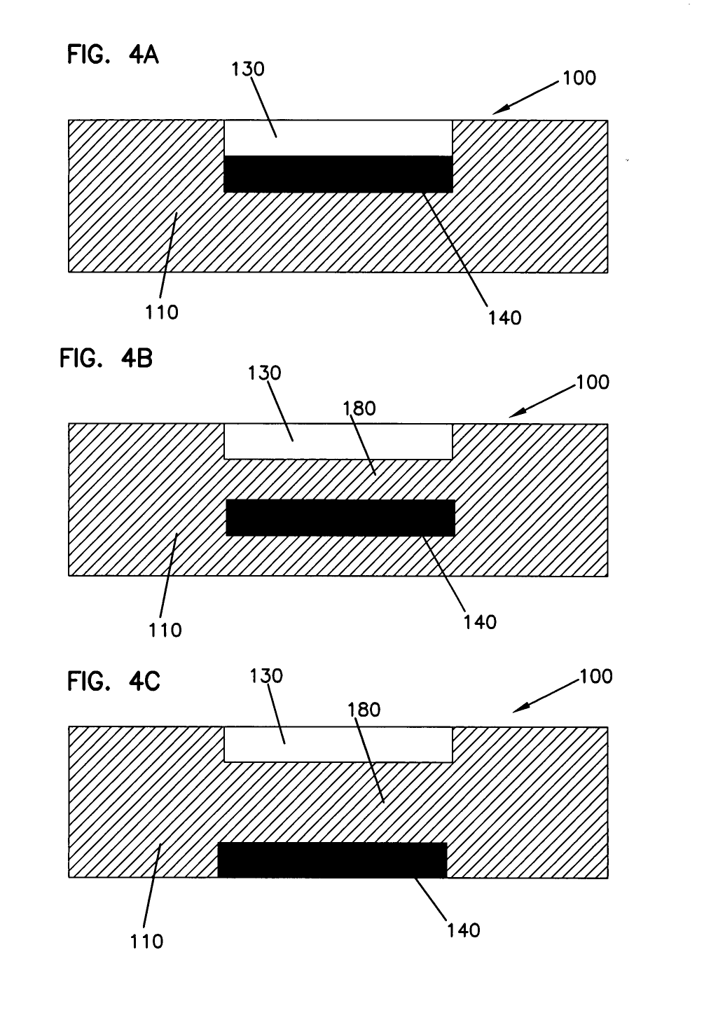

In some embodiments, the thermal control device 140 is embedded in the substrate 110 of the microfluidic nucleic acid assay device 100. Referring to FIGS. 4A-4C, cross-sectional views of a portion of microfluidic nucleic acid assay device 100 are shown. Visible in the cross-sectional view of FIG. 4A is substrate 110 and reaction region 130. Also visible is thermal control device 140, which is hidden from view in FIG. 1 because it is disposed underneath reaction region 130. In FIG. 4A, thermal control device 140 is disposed so as to be in direct contact with a sample disposed in reaction region 130.

In FIG. 4B, a similar embodiment to FIG. 4A is shown, except that thermal control device 140 is disposed proximate to, but not in direct contact with, the bottom of reaction region 130 and is separated from reaction region 130 defining spacer 180. In this embodiment, a sample placed in reaction region 130 does not directly contact thermal control device 140 because of spacer 180.

In FIG. 4C, a similar embodiment to FIG. 4B is shown, except that thermal control device 140 is disposed at the side of substrate 110 opposite the side in which reaction region 130 is defined. Thus, the spacer 180 defined in this embodiment is larger than spacer 180 of FIG. 4B, and exposes thermal control device 140 to the external surface of the substrate 110 of the microfluidic nucleic acid assay device 100. Exposure of thermal control device 140 to the external surface of the substrate 110 of microfluidic nucleic acid assay device 100 provides convenient access to the thermal control device such that a source of electricity is easily connected to it.

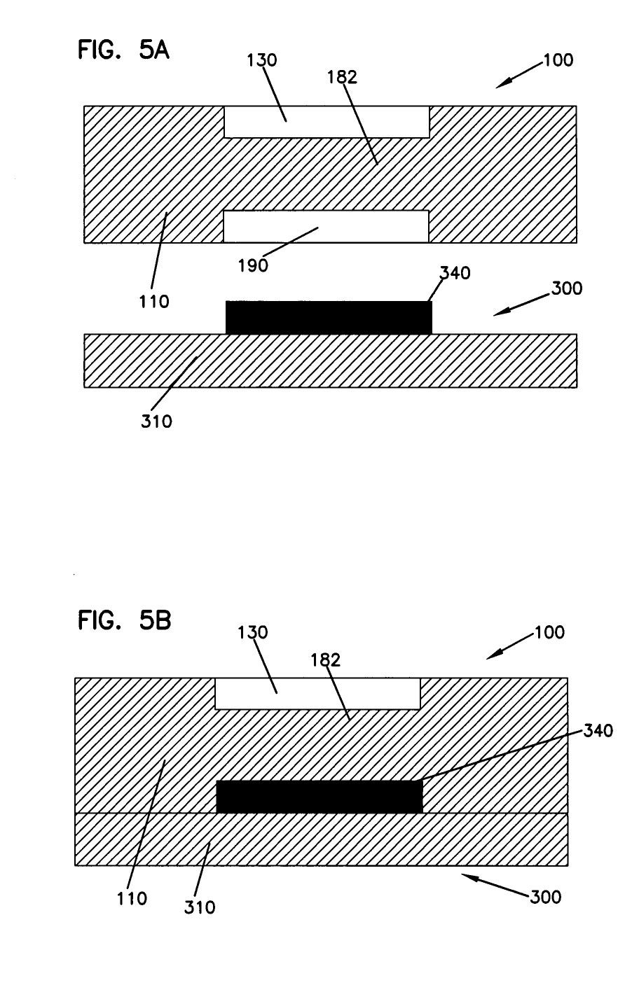

In some embodiments, thermal control device 140 is situated external to the microfluidic nucleic acid assay device 100. Referring to FIGS. 5A and 5B, cross-sectional views of a microfluidic nucleic acid assay device 100 and a thermal module 300 are shown. A recess 190 is defined in substrate 110 in the side of the substrate 110 opposite the side in which reaction region 130 is defined and proximate to reaction region 130. Recess 190 is a hollowed out area of substrate 110 corresponding to the dimensions of thermal control device 340. Recess 190 is thus shaped to receive thermal control device 340. Thermal control device 340 is situated on substrate 310 of thermal module 300. Together, recess 190 and reaction region 130 define spacer 182. In these embodiments, when the microfluidic nucleic acid assay device 100 is situated within a machine having thermal control device 340, thermal control device 340 is situated within recess 190 proximate to reaction region 130.

In some embodiments, thermal control device 140, 340 is a thermoelectric device. In other embodiments, thermal control device 140, 340 is a resistive device (joule heater). In some embodiments, thermal control device 140, 340 is a microheater chip. In other embodiments, thermal control device 140, 340 is an electromagnetic heating device, for example, an infrared emitting device or a microwave emitting device disposed above or below the side of substrate 110 in which reaction region 130 is defined.

In embodiments where the electromagnetic heating device is a microwave emitting device, water present in the nucleic acid sample converts microwave radiation emitted by the microwave emitting device to heat. Where the electromagnetic heating device is an infrared emitting device, the microfluidic nucleic acid assay device 100 additionally includes a structure, such as a black body, that absorbs the infrared radiation emitted by the infrared emitting device and converts the absorbed radiation into heat. One example of a suitable infrared emitting device is an infrared laser. Additionally or alternatively, the electromagnetic heating device may emit electromagnetic energy in other regions of the electromagnetic spectrum, e.g., visible light.

In embodiments such as those shown in FIGS. 4B-4C or FIGS. 5A-5B, thermal control device 140, 340 can be situated about 25 μm to 500 μm from a bottom portion of reaction region 130. In other embodiments, thermal control device 140, 340 can be situated about 100 μm from a bottom portion of the reaction region 130.

In some embodiments thermal control device 140, 340 is in the form of a ring that surrounds the sample well. In other embodiments thermal control device 140, 340 is in the form of a cylinder having one closed end, such that thermal control device 140, 340 encloses reaction region 130, 230. In other embodiments thermal control device 140, 340 is circular, oval, or rectilinear in shape. In some embodiments, thermal control device 140, 340 matches one or more dimensions of reaction region 130, 230. In other embodiments thermal control device 140, 340 is smaller than one or more dimensions of reaction region 130, 230. In still other embodiments thermal control device 140, 340 is larger than one or more dimensions of reaction region 130, 230.

In some embodiments thermal control device 140, 340 is disposed above the reaction region 130. Such embodiments can include electromagnetic thermal control devices 140, 340, for example microwave or infrared heating devices. In other embodiments, thermal control device 140, 340 is disposed below the well. In still other embodiments, thermal control device 140, 340 is disposed on the side of the well.

In embodiments thermal control device 140, 340 is capable of thermally cycling a nucleic acid sample in reaction region 130 to perform a biochemical reaction. In other embodiments, thermal control device 140, 340 is capable of thermally cycling a nucleic sample in reaction region 130 to perform a polymerase chain reaction. In other embodiments, thermal control device 140, 340 is capable of providing a series of incubation temperatures to a nucleic acid sample in the reaction region 130, wherein the incubation temperatures are suitable to perform a restriction enzyme digestion.

In some embodiments, the thermal control device 140, 340 is capable of heating a sample placed in the reaction region 130 to a target temperature of up to about 90° C. or 105° C. In other embodiments, the thermal control device 140, 340 is capable of heating a sample placed in the reaction region 130 to the target temperature at a rate of about 1° C./sec to 10° C./sec. In still other embodiments, the thermal control device 140, 340 is capable of maintaining the target temperature of a sample place in reaction region 130 to within about 0.1° C. to 2° C. In some embodiments, the thermal control device 140, 340 is a thermoelectric device capable of removing heat from a sample placed in the reaction region 130 at a rate of about 1° C./sec to about 10° C./sec.

Nucleic Acid Separation Region

Various chromatographic techniques of separating molecular species using a microfluidic device are disclosed in U.S. Pat. Nos. 7,128,876, 6,702,256, and 6,958,119, which are incorporated herein by reference in their entirety. In some embodiments, the nucleic acid separation region 150 is a column having a length of about 0.5 cm to about 5 cm.

Nucleic acid separation region 150 includes one or more solid phase media situated to facilitate separation of nucleic acid samples. A solid phase medium may include one or more of several different materials. The choice of a solid phase medium depends on the nucleic acid sample being separated and the type of separation employed. Embodiments are not limited by the choice or number of solid phase media disposed in the nucleic acid separation region 150. The choice of a solid phase medium depends on, for example, whether nucleic acid separation region 150 separates nucleic acid samples by size exclusion chromatography, high pressure liquid chromatography, or electrophoresis. The choice of a solid phase medium also depends on elution conditions such as temperature, buffer material, amount of pressure applied, and similar considerations that will be readily identified by the user.

In some embodiments, the nucleic acid separation region 150 of microfluidic nucleic acid assay device 100 is an electrophoretic separation column. In embodiments where the nucleic acid separation region 150 is an electrophoretic separation column, the electrophoretic separation column includes, in embodiments, solid phase media such as poly-N,N-dimethylacrylamide, methylcellulose, hydroxyethylcellulose, hydroxypropylcellulose, hydroxypropylmethylcellulose, dextran, linear polyacrylamide, poly-N-acryloylaminoethoxyethanol, polyacryloylaminopropanol, poly(acryloylaminoethoxy)ethyl-glucopyranoside, poly(ethylene glycol), poly(ethylene oxide), poly(vinyl pyrrolidone), agarose, or a mixture of one or more thereof. In some embodiments, the solid phase in the microfluidic channels includes a thermoresponsive polymer. Thermoresponsive polymer are discussed above, under the heading “Substrate”. Any known media used to carry out electrophoretic separation of nucleic acids are useful in such embodiments; the electrophoretic separation column is not limited by the type of medium used to facilitate separation of nucleic acids.

The material can be a crosslinked polymer. Any known medium used to carry out electrophoretic separation is useful in such embodiments; the electrophoretic separation column is not limited by the type of medium used to facilitate separation of nucleic acids. It will be understood that the separation medium is selected depending on the type and/or molecular weight of the nucleic acid to be separated.

In embodiments where nucleic acid separation region 150 is an electrophoretic separation column, the solid phase medium contains one or more buffers. Examples of buffers commonly employed in embodiments are tris-HCl, tris-acetate, tris-phosphate, tris-borate, sodium hydroxide, urea, glycine, EDTA, or mixtures of these. The buffers can be employed over a range of pH. In embodiments, the pH of the buffer solution is adjusted to about 7 to 9, about 7.5 to 8, or about 7.5 to 7.8.

In some embodiments where nucleic acid separation region 150 is an electrophoretic separation column, the solid phase medium is an agarose gel. In such embodiments, agarose is present in the electrophoretic separation column at about 0.1 w/v % to 10 w/v %, about 0.3 w/v % to 2 w/v %, or about 1 w/v % in the gel. In other embodiments, the solid phase medium is a polyacrylamide gel. In such embodiments, the polyacrylamide is crosslinked. Typically, polyacrylamide gel is crosslinked by N,N-methylene bisacrylamide, although other crosslinkers may be used. In embodiments, the degree of crosslinking of the polyacrylamide gel is about 1 crosslink per 29 acrylamide repeat units. In embodiments, the concentration of polyacrylamide of the solid phase medium is about 3 w/v % to 25 w/v %, or about 5 w/v % to 20 w/v %. In embodiments, the polyacrylamide gel is a nondenaturing polyacrylamide gel. In other embodiments, the polyacrylamide gel is a denaturing polyacrylamide gel. Denaturing polyacrylamide gels include, in embodiments, denaturing agents urea, formamide, or a combination thereof. In embodiments, the denaturing polyacrylamide gel includes a gradient of denaturing agent

In embodiments where nucleic acid separation region 150 is an electrophoretic separation column, the nucleic acid separation region 150 has a first end and a second end, wherein the first end is in fluid connection with the reaction region 130, and the second end is where the sample ends up after elution through nucleic acid separation region 150. In some embodiments, substrate 110 of microfluidic nucleic acid assay device 100 defines an additional well in fluid communication with the second end of the nucleic acid separation region 150 to collect fluids issuing from the nucleic acid separation region 150.

In embodiments, the nucleic acid separation region 150 of the microfluidic nucleic acid assay device includes a selected length, width, and depth. In embodiments, the nucleic acid separation region 150 is about 75 mm to 1 mm long, about 50 mm to 5 mm long, or about 25 mm to 10 mm long. In embodiments, the nucleic acid separation region 150 is about 2 μm to 25 μm deep, about 7 μm to 18 μm deep, or about 11 μm to 14 μm deep. In embodiments, the nucleic acid separation region 150 is about 5 μm to 100 μm wide, about 20 μm to 80 μm wide, or about 30 μm to 50 μm wide.

For example, a longer nucleic acid separation region 150 typically allows for finer resolution of nucleic acids eluting through the region, while a shorter nucleic acid separation region 150 typically allows faster elution but with less resolution.

In embodiments where the nucleic acid separation region is an electrophoretic separation column, the microfluidic nucleic acid assay device 100 has two or more electrodes situated to apply an electrical voltage across the electrophoretic separation column. The electrical voltage, when applied, serves to urge a nucleic acid sample through the electrophoretic separation column, thereby separating the nucleic acid into individual molecular species. In embodiments, the electrical voltage is applied in a pulsed field. In some such embodiments, the electrical field is pulsed and alternated orthogonally across the solid phase medium. In other embodiments, the electrical field is inverted periodically, such that each inversion represents a pulse. In such embodiments, the length of one pulse is set to be longer than the other pulse.

Referring to FIG. 2, in some embodiments, nucleic acid separation region 250 of microfluidic nucleic acid assay device 200 is has a first end and a second end, wherein the first end is in fluid connection with reaction region 230. Microfluidic nucleic acid assay device 200 has well 224 in fluid communication with the second end of the nucleic acid separation region 150 to collect separated nucleic acids issuing therefrom.

In embodiments, nucleic acid separation region 150, 250 is defined in substrate 110 by a technique. Techniques include lithography, laser etching, printing, and microreplication of inverse images of features disposed on PDMS, nickel, or polyimide onto a final substrate such as a thermoplastic. In some embodiments, microreplication is carried out by a melt techniques, such as applying heat and pressure to a thermoplastic disposed against a mold having an inverse pattern. In other embodiments, microreplication can be carried out by casting an uncured polymer such as PDMS onto a mold and curing the polymer, followed by removal of the mold.

In other embodiments, nucleic acid separation region 150, 250 is defined in substrate 110 by etching a glass substrate using lithographic techniques. Any of these techniques, or others, can be used to define nucleic acid separation region 150, 250 on substrate 110 of microfluidic nucleic acid assay device 100. The described techniques are merely illustrative and do not limit the techniques that can be used to define features on substrate 110 of microfluidic nucleic acid assay device 100.

Valves

In embodiments, substrate 110 of microfluidic nucleic acid assay device 100 defines one or more valves 162, 164. In other embodiments, substrate 210 of microfluidic device 200 defines valves 260, 262, 264. The valves of microfluidic nucleic acid assay devices 100, 200 are, in embodiments, mechanical valves such as those described in U.S. Pat. No. 6,702,256, which is incorporated herein by reference in its entirety.

In other embodiments, microfluidic nucleic acid assay devices 100, 200 have valves including a thermoelectric device capable of providing either addition of heat or removal of heat, and a first material capable of phase change upon addition of heat or removal of heat. The first material capable of phase change can be a sample which is placed in the fluid connection between well 122, 222 and reaction region 130, 230, such as in channel 272. The first material can also be a separate material that is a layer on at least a portion of substrate 110 and that is thermoresponsive, such that it can change phase when the first thermoelectric device removes heat from the area proximal to first thermoelectric device. Such thermoresponsive materials, e.g. thermoresponsive polymers, are described in the section above entitled “Substrate.” In these embodiments, the thermoelectric device of the valve is made to remove heat in order to “close” the valve. Upon removal of heat, the material capable of phase change forms a solid phase within the fluid communication between sample well 122, 222 and reaction region 130, 230, such as in channel 272. The solid phase, in turn, creates a barrier to materials moving between sample well 122, 222 and reaction region 130, 230. The barrier constitutes the valve in such embodiments. The valve is then “opened” by adding heat via the thermoelectric device sufficient to melt the phase change material and reopen the fluid communication between sample well 122, 222 and reaction region 130, 230.

In embodiments, microfluidic nucleic acid assay device 100 has second valve 164 including a second thermoelectric device capable of providing either addition of heat or removal of heat, and a second material capable of phase change upon addition of heat or removal of heat. The second material capable of phase change can be a sample which is placed in the fluid connection between reaction region 130 and nucleic acid separation region 150. The second material capable of phase change can also be a separate material that is a layer on at least a portion of substrate 110 and that is thermoresponsive, such that it can change phase when the second thermoelectric device removes heat from the area proximal to second thermoelectric device.

Valves employing thermoelectric devices and phase changing materials are described in U.S. Pat. Nos. 6,007,302 and 5,975,856, which are incorporated herein by reference in their entirety.

In embodiments, valves 164, 264 are opened intermittently during amplification. In embodiments, valves 164, 264 are opened between each repetition of the amplification cycle. In some embodiments, valves 164, 264 are opened between every other repetition of the amplification cycle. In some embodiments, valves 164, 264 are opened 5 to 15 times during the amplification cycle. In embodiments, when valves 164, 264 are opened, portions of amplified nucleic acid from the reaction region 130, 230 is urged to nucleic acid separation region 150, 250 and separated. The portions of amplified nucleic acid have, in embodiments, a volume of about 10 picoliters to 1 nanoliter.

Channels

In some embodiments, fluid connections between features of microfluidic nucleic acid assay device 100 are channels. Referring to FIG. 2, for example, substrate 210 of microfluidic nucleic acid assay device 200 defines a channel 272 disposed between the sample wells 222 and the reaction region 230. In other embodiments, a channel 274 connects reaction region 230 and nucleic acid separation region 250. In other embodiments, channels 270 connect one or more additional wells 220 to other features defined on substrate 210.

In embodiments, channels 270, 272, and 274 are microfluidic channels. Typically, in embodiments, channels 270, 272, and 274 have a width of about 10 micrometers to about 100 micrometers and a depth of about 5 micrometers to about 50 micrometers.

Microfluidic channels 270, 272, 274 are defined on substrate 210 by employing a technique. Techniques include lithography, laser etching, printing, and microreplication of inverse images of features disposed on PDMS, nickel, or polyimide onto a final substrate such as a thermoplastic. In some embodiments, microreplication is carried out by a melt technique, such as applying heat and pressure to a thermoplastic disposed against a mold having an inverse pattern. In other embodiments, microreplication can be carried out by casting an uncured polymer such as PDMS onto a mold and curing the polymer, followed by removal of the mold. In other embodiments, features are etched onto a glass substrate using a lithographic technique. Any of these techniques, or others, can be used to form microfluidic channels 270, 272, 274 on substrate 210 of microfluidic nucleic acid assay device 200. The described techniques are merely illustrative and do not limit the techniques that can be used to make microfluidic nucleic acid assay device 200.

Reagents

In embodiments, microfluidic nucleic acid assay device 100 includes a source of reagents. In some embodiments the reagents are preloaded into the microfluidic nucleic acid assay device 100 prior to addition of a nucleic acid sample. Such reagents are, in embodiments, reagents suitable for nucleic acid annealing, nucleic acid amplification, nucleic acid detection, or a combination of these. The reagents include, in some embodiments, one or more buffer solutions, dyes, gels, standards, templates, primers, enzymes, and/or nucleotides.

Referring to FIG. 2, in embodiments, one or more reagents can be disposed in one or more additional wells 220. In embodiments, the one or more additional wells 220 are in fluid communication with at least one of the sample well 222, reaction region 230, and/or nucleic acid separation region 250. In some embodiments, one or more additional wells 220 are in fluid connection with the sample well 222. In other embodiments one or more additional wells 220 are in fluid communication with reaction region 230. In other embodiments one or more additional wells 220 are in fluid communication with nucleic acid separation region 250. In yet other embodiments one or more additional wells 220 are in fluid communication with more than one of sample well 222, reaction region 230, and nucleic acid separation region 250.

In embodiments, one or more buffer solutions are present in one or more additional wells 220. The choice of buffer employed with the microfluidic nucleic acid assay device 200 is not particularly limited and should be chosen based on the nucleic acid being assayed. Examples of buffers commonly employed in embodiments are tris-HCl, tris-acetate, tris-phosphate, tris-borate, sodium hydroxide urea, glycine, or mixtures of these at various values of pH (Sigma-Aldrich Company of St Louis, Mo.). In some embodiments, the buffer solution provides a suitable chemical environment for activity and stability of nucleic acid polymerase and/or other reagents for annealing and amplification of nucleic acids. In some embodiments, the buffer is used to maintain a constant concentration of the nucleic acid. In embodiments, a buffer is used to transport one or more other reagents within the microfluidic nucleic acid assay device 200. In other embodiments, the buffer solution provides for elution the nucleic acid sample within the nucleic acid separation region 250; in still other embodiments it is used for a combination of these purposes.

In embodiments, the one or more templates are nucleic acid templates containing one or more regions of nucleic acid fragments to be amplified. In embodiments, the one or more primers, which are complementary to the nucleic acid regions at the 5′ and 3′ ends of the region of the nucleic acid that is to be amplified. In embodiments, the one or more enzymes include a nucleic acid polymerase, used to synthesize a nucleic acid copy of the one or more regions to be amplified. In embodiments, the one or more nucleotides include nucleotide triphosphates, from which the nucleic acid polymerase builds one or more new nucleic acids.

System

In embodiments, the microfluidic nucleic acid assay device 100 is one part of a system that facilitates the amplification and separation of nucleic acids. For example, a microfluidic nucleic acid assay device may be inserted into an instrument, such as the Agilent 2100 Bioanalyzer by Agilent Technologies, Inc. of Santa Clara, Calif. The instrument, in embodiments, facilitates one or more operations associated with assaying nucleic acids. The microfluidic nucleic acid assay device 100 is not limited by the choice of instrumentation employed.

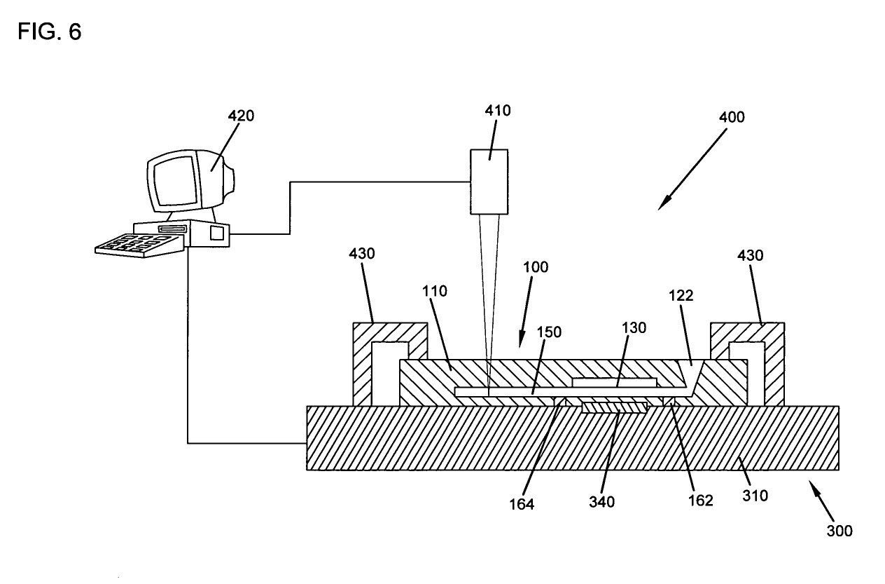

Referring to FIG. 6, a system 400 is shown. System 400 incorporates microfluidic nucleic acid assay device 100 having substrate 110. Defined in substrate 110 are features including sample well 122, reaction region 130, nucleic acid separation region 150, first valve 162 disposed between sample well 122 and reaction region 130, and second valve 164 disposed between reaction region 130 and nucleic acid separation region 150. Valves 162, 164 are thermoelectric elements. Also shown is thermal module 300, having module substrate 310 and thermal control device 340 operatively connected to module substrate 310. System 400 further includes an optical interrogation module 410, which analyzes a nucleic acid sample as it progresses through nucleic acid separation region 150. In some embodiments, system 400 includes an external computer 420. For example, external computer 420 can be in data connection at least with thermal module 300 and optical interrogation module 410. System 400 further includes clamps 430 to provide a secure connection between microfluidic nucleic acid assay device 100 and thermal module 300.

In some embodiments of a system 400 for amplification and separation of nucleic acids, an instrument facilitates one or more operations of the amplification or separation of nucleic acids. In embodiments, the instrument receives one or more signals from a sample or a device such a microfluidic nucleic acid assay device 100 or thermal control module 300. In some embodiments, external computer 420 is present as part of an instrument in data communication with one or more features of microfluidic nucleic acid assay device 100 or system 400, for example with valves 162, 164 or thermal control device 340. In embodiments, a data connection exists between external computer 420 and thermal control device 340 that is located on an instrument. In other embodiments, a data connection exists between computer 420 and a thermal control device embedded within the microfluidic nucleic acid assay device 100. In some embodiments external computer 420 interprets data in the form of signals arising from thermal control device 340, from analysis of separated nucleic acids by optical interrogation module 410, or from some other part of system 400. In some embodiments external computer 420 provides signals to control the heating or cooling of the sample in the reaction region by providing data to control a switch that switches current to the thermal control device 340 on and off. In other embodiments external computer 420 switches the direction of current applied to the thermal control device 340, or otherwise controls the amount and direction of current applied to the thermal control device 340.

In some embodiments of a system 400 for amplification and separation of nucleic acids, external computer 420 is in data communication with one or more valves 162, 164 present on the microfluidic nucleic acid assay device 100. In embodiments, external computer 420 provides control signals to control the heating or cooling of by a thermoelectric device of one or more valves 162, 164. The thermoelectric devices of valves 162, 164 are heated or cooled sufficiently to provide a phase change in a material that is capable of phase change upon addition of heat or removal of heat. In embodiments, the control is provided by a current that is applied to the thermoelectric devices of valves 162, 164. In some embodiments, one or both of the first valve 162 and the second valve 164 are closed prior to the amplifying of a nucleic acid sample.

In other embodiments of system 400, second valve 164 is opened at the end of each repeating heat cycle during amplification, and second valve 164 is closed again prior to initiating the subsequent heat cycle. This embodiment enables portions of an amplified nucleic acid to be removed during amplification. The portions of amplified nucleic acid have, in embodiments, a volume of about 10 picoliters to 1 nanoliter. Real time sampling of the nucleic acid sample during amplification is thereby enabled. In some embodiments, the portions of amplified nucleic acid are urged toward the nucleic acid separation region 150 after each opening of the second valve 164 and are separated in the nucleic acid separation region 150.

In some embodiments of a system 400 for amplification and separation of nucleic acids, a sample including nucleic acids moves through the nucleic acid separation region 150 of the microfluidic nucleic acid assay device 100 by pressure, electrophoresis, or a combination thereof. In some embodiments, the pressure and electrophoresis are supplied to the nucleic acid separation region 150 of the microfluidic nucleic acid assay device 100 by an instrument in which the microfluidic nucleic acid assay device is inserted, for example, the Agilent 2100 Bioanalyzer® by Agilent Technologies, Inc. of Santa Clara, Calif. In some embodiments, the instrument provides both pressure and electrophoretic mobility, individually or simultaneously, to the nucleic acid separation region 150 of the microfluidic nucleic acid assay device 100. In some embodiments, external computer 420 is in data communication with one or more nucleic acid separation regions 150, one or more sources of pressure or electrophoretic mobility, or any combination thereof. In embodiments, external computer 420 interprets data signals from the nucleic acid separation region. In other embodiments, external computer 420 provides signals to one or more sources of pressure or electrophoretic mobility in order to control movement of a sample through one or more nucleic acid separation regions 150.

In some embodiments of a system 400 for amplification and separation of nucleic acids, an instrument includes a detector source, such as an optical interrogation module 410. Optical interrogation module 410 can provide an optical signal to a separated amplified nucleic acid sample eluting through nucleic acid separation region 150. In embodiments, the interaction of the optical signal with the eluting separated amplified nucleic acid sample provides a means of analysis of the sample. For example, the result of the interaction of the optical signal and the eluting sample may be a modified signal, which is detected by a detection apparatus (not shown). In embodiments, a detected signal is sent to external computer 420, which interprets the modified signal and presents results in readable form for a human user. In some embodiments, the optical interrogation module 410 provides a signal that interacts with a sample to provide an ultraviolet measurement, an infrared measurement, or a fluorescence measurement.

The various embodiments described above are provided by way of illustration only and should not be construed to limit the claims attached hereto. Various modifications and changes may be made without following the example embodiments and applications illustrated and described herein, and without departing from the scope of the following claims.

The present invention may suitably comprise, consist of, or consist essentially of, any of the disclosed or recited elements. Thus, the invention illustratively disclosed herein can be suitably practiced in the absence of any element which is not specifically disclosed herein.

What is claimed:

1. A microfluidic nucleic acid assay device comprising a substrate defining features, the features comprising:
a sample well,
a reaction region in fluid connection to the sample well,
a thermal control device positioned proximal to the reaction region,
a nucleic acid separation region in fluid communication with the reaction region,
a first valve disposed between the well and the reaction region, and
a second valve disposed between the reaction region and the nucleic acid separation region;
wherein the features collectively occupy an area of the substrate of about 0.1 to 10 cm².

2. The microfluidic nucleic acid assay device of claim 1 wherein the substrate comprises glass, silicon, or poly(ether ether ketone).

3. The microfluidic nucleic acid assay device of claim 2 wherein the substrate further comprises a thermoresponsive polymer.

4. The microfluidic nucleic acid assay device of claim 3 wherein the thermoresponsive polymer comprises poly(ethylene oxide-block-propylene oxide), poly(isopropylacrylamide-graft-poly(ethylene oxide)), poly(acrylamide-graft-poly(isopropylacrylamide)), a blend of hydroxypropylcellulose and hydroxyethylcellulose, a blend of N,N-dialkylacrylamide copolymers, poly(N,N-dimethylacrylamide-graft-poly(ethylene oxide)), or a blend thereof.

5. The microfluidic nucleic acid assay device of claim 1 wherein:
the first valve comprises
a first thermoelectric device capable of providing either addition of heat or removal of heat, and
a first material capable of phase change upon addition of heat or removal of heat; and
the second valve comprises
a second thermoelectric device capable of providing either addition of heat or removal of heat, and
a second material capable of phase change upon removal or addition of heat.

6. The microfluidic nucleic acid assay device of claim 5 wherein
the fluid connection between the sample well and the reaction region comprises a first channel wherein the first thermoelectric device is proximate to the first channel; and
the fluid communication between the reaction region and the nucleic acid separation region comprises a second channel, wherein the second thermoelectric device is proximate to the second channel.

7. The microfluidic nucleic acid assay device of claim 1 wherein the thermal control device comprises one of a thermoelectric device, a resistive device, and an electromagnetic device.

8. The microfluidic nucleic acid assay device of claim 1 wherein the thermal control device is embedded in the substrate.

9. The microfluidic nucleic acid assay device of claim 1 wherein the thermal control device is situated about 25 μm to 500 μm from a bottom portion of the reaction region.

10. The microfluidic nucleic acid assay device of claim 1 wherein the thermal control device is situated about 100 μm from a bottom portion of the reaction region.

11. The microfluidic nucleic acid assay device of claim 1 wherein the reaction region can hold a sample having a volume of about 1 picoliter to 25 microliters.

12. The microfluidic nucleic acid assay device of claim 1 wherein:
the thermal control device is capable of thermally cycling a sample in the reaction region, and
the thermal cycling is suitable to perform a biochemical reaction comprising a polymerase chain reaction.

13. The microfluidic nucleic acid assay device of claim 1 wherein:
the thermal control device is capable of providing a series of incubation temperatures to a sample in the reaction region, and
the incubation temperatures are suitable to perform a biochemical reaction comprising a restriction enzyme digestion.

14. The microfluidic nucleic acid assay device of claim 1 wherein the nucleic acid separation region comprises an electrophoretic separation column.

15. The microfluidic nucleic acid assay device of claim 1 further comprising a source of reagents, the reagents comprising one or more of a buffer, a template, a primer, an enzyme, and a nucleotide, wherein:
the sample well is a first well,
the features further comprise additional wells,
the additional wells are in fluid communication with at least one of the sample well, the reaction region and the nucleic acid separation region; and
the reagents are disposed within the additional wells.

16. A method of amplifying and separating nucleic acids, the method comprising:
providing a microfluidic nucleic acid assay device comprising a substrate defining features, the features comprising
a sample well,
a reaction region in fluid connection to the well,
a thermal control device positioned proximal to the reaction region,
a nucleic acid separation region in fluid communication with the reaction region,
a first valve disposed between the sample well and the reaction region, and
a second valve disposed between the reaction region and the nucleic acid separation region;
wherein the features collectively occupy an area of the substrate of about 0.1 to 10 cm²;
injecting a nucleic acid sample into the sample well;
urging the nucleic acid sample into the reaction region;
amplifying the nucleic acid sample by a polymerase chain reaction; and
separating the amplified nucleic acid sample.

17. The method of claim 16 wherein the amplifying comprises:
a first heating comprising heating the nucleic acid sample to about 94° C. to 96° C. for about 1 to 9 minutes,
an annealing comprising allowing the nucleic acid sample to cool to about 40° C. to 64° C.,
holding of the nucleic acid sample temperature at about 50° C. to 64° C. for about 20 to 40 seconds,
a second heating comprising heating the nucleic acid sample to about 65° C. to 75° C. for about 20 to 40 seconds,
a third heating comprising heating the nucleic acid sample to about 94° C. to 98° C. for about 20 to 30 seconds,
repeating the annealing, the holding, the second heating, and the third heating between 10 and 35 times, and
maintaining of the temperature of the nucleic acid sample at about 70° C. to 75° C. for about 5 to 15 minutes.

18. The method of claim 16 further comprising analyzing the separated amplified nucleic acid sample.

19. The method of claim 18, wherein the analyzing is accomplished optically.

20. The method of claim 19 wherein the analyzing comprises one of an ultraviolet measurement, an infrared measurement, and a fluorescent measurement.

21. The method of claim 17 further comprising closing the first valve and the second valve prior to the amplifying.

22. The method of claim 21 further comprising opening the second valve at the end of each repeating and closing the second valve prior to a subsequent repeating.

23. The method of claim 22 further comprising separating a portion of an amplified nucleic acid sample after each repeating.

24. The method of claim 23 wherein the portion of an amplified nucleic acid sample comprises a volume of about 10 picoliters to 1 nanoliter.
